(19) United States
(12) Patent Application Publication
(10) Pub. No.: US 2024/0408641 A1
(43) Pub. Date: Dec. 12, 2024

(54) PAINT ROLLER WITH RELEASE MECHANISM

(71) Applicant: LEG UP, Marietta, GA (US)

(72) Inventor: Clinton Smith, Marietta, GA (US)

(73) Assignee: LEG UP, Marietta, GA (US)

(21) Appl. No.: 18/330,793

(22) Filed: Jun. 7, 2023

(51) Int. Cl. B05C 17/02

(52) U.S. Cl. CPC B05C 17/0217; B05C 17/0205

(57) ABSTRACT

An apparatus having a roller assembly and a trigger mechanism is disclosed. The roller assembly may include a first collar disposed at a roller assembly proximal end and a second collar disposed at a roller assembly distal end. The roller assembly may further include a guide rod having a guide rod proximal end and a guide rod distal end. The guide rod distal end may be attached to the second collar, and the guide rod may be disposed between the first collar and the second collar. The roller assembly may further include a plurality of deformable rods disposed between the first collar and the second collar. The plurality of deformable rods may be configured to move between a tensioned state and an un-tensioned state. The apparatus may further include a trigger mechanism configured to move the plurality of deformable rods between the tensioned state and the un-tensioned state.

### TECHNICAL FIELD

The present disclosure relates to a paint roller, and more specifically to a paint roller having a release mechanism to release a roller cover or a paint brush from the paint roller.

### BACKGROUND

A paint roller is used to apply paint or coating to large surfaces rapidly and efficiently. A paint roller typically includes a roller assembly on which a roller cover or a paint brush may be disposed or inserted. The roller cover is generally removable and can be removed and cleaned or discarded after use.

While using a conventional paint roller, a user is typically required to grasp the roller cover from its outer surface and pull the roller cover out of the roller assembly, when the user desires to replace or remove the roller cover. As the roller cover outer surface may be covered with wet paint, the process of removing the roller cover by pulling it from the roller assembly may be messy and may cause inconvenience to the user.

Thus, there is a need for a mechanism that may facilitate the user to conveniently remove the roller cover from the roller assembly.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1A depicts an example paint roller in a tensioned state in accordance with the present disclosure.

FIG. 1B depicts the paint roller of FIG. 1A in an un-tensioned state in accordance with the present disclosure.

FIG. 2 depicts the paint roller of FIGS. 1A, 1B with a roller cover in accordance with the present disclosure.

FIG. 3 depicts another paint roller in accordance with the present disclosure.

FIG. 4 depicts yet another paint roller in accordance with the present disclosure.

FIG. 5A depicts an example paint roller in an unlocked state in accordance with the present disclosure.

FIG. 5B depicts the paint roller of FIG. 5A in a locked state in accordance with the present disclosure.

### DETAILED DESCRIPTION

### Overview

The present disclosure is directed towards a paint roller that may enable a user to conveniently remove/replace a roller cover. The paint roller may include a roller assembly, a roller frame and a handle. The roller cover may be disposed on the roller assembly. The roller assembly may include a first collar and a second collar that may be disposed at opposite ends of the roller assembly. The roller assembly may further include a guide rod and a plurality of deformable rods disposed between the first collar and the second collar. The paint roller may further include a trigger mechanism that may be configured to change a distance between the first collar and the second collar by laterally sliding the guide rod. Changing distance between the first collar and the second collar may move the plurality of deformable rods between a tensioned state and un-tensioned state. The plurality of deformable rods may be configured to secure the roller cover on the roller assembly in the tensioned state.

In another embodiment, the paint roller may include a “Y-shaped” roller frame that may include a moveable arm and a stationary arm. The paint roller may further include supporting members that may be connected to the moveable arm and the stationary arm. The supporting members may be configured to support the roller cover on the paint roller. The paint roller may further include a trigger mechanism (such as a dial index mechanism) that may be configured to move the moveable arm to attach/detach respective supporting members, thereby facilitating easy removal/replacement of the roller cover.

The present invention discloses a paint roller that may facilitate easy removal/replacement of the roller cover without requiring to touch the roller cover during the removal process. Specifically, the present disclosure facilities the user to replace a wet roller cover automatically, thereby preventing user's hands from getting messy or wet by paint/coating that may be present on the roller cover. Further, the paint roller is simple in design and easy to manufacture.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

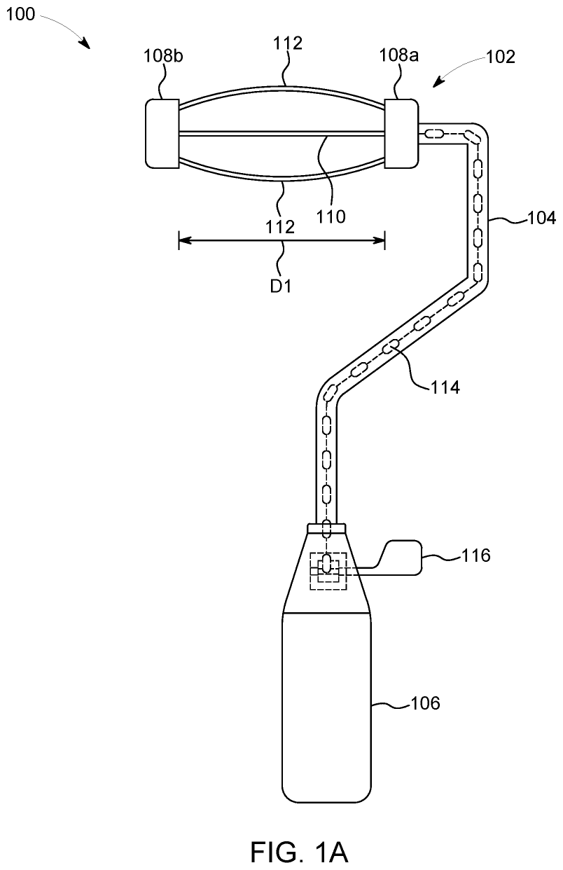

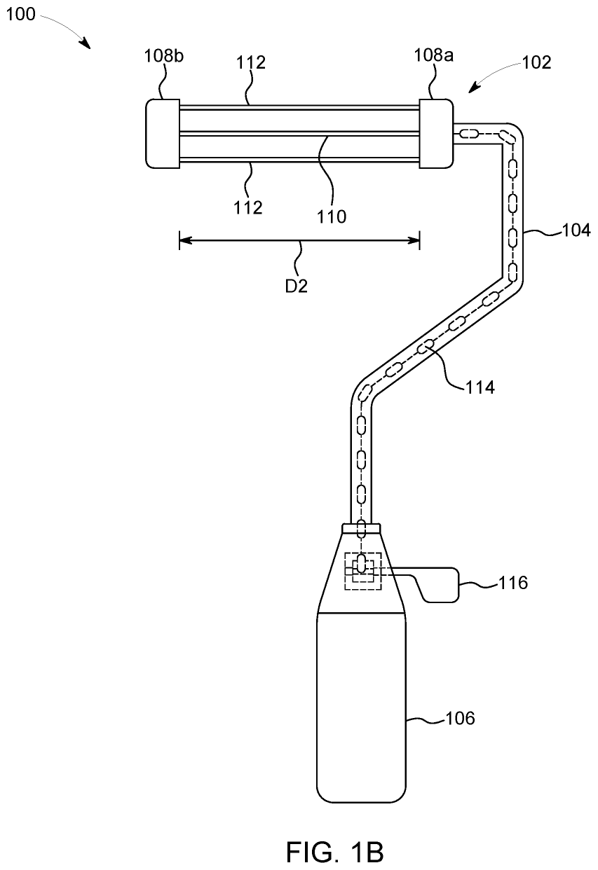

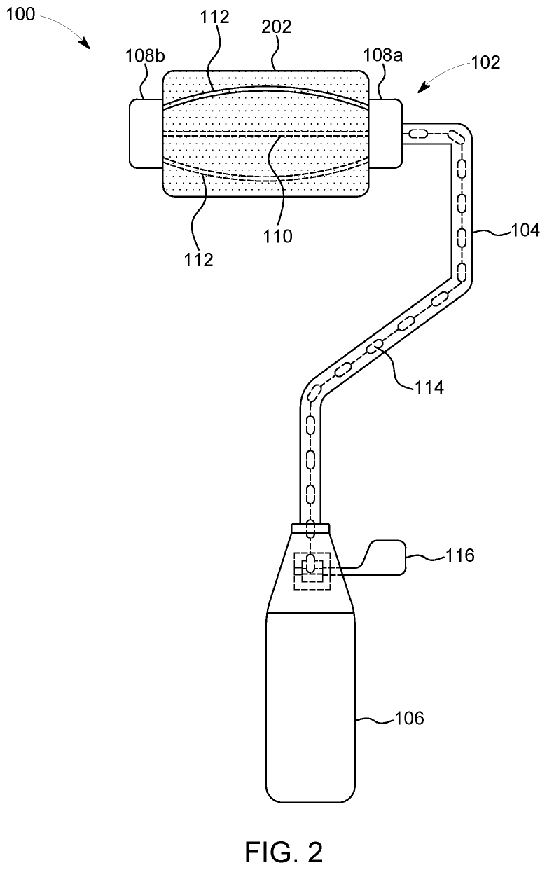

FIG. 1A depicts an example paint roller 100 in a tensioned state in accordance with the present disclosure, and FIG. 1B depicts the paint roller 100 in an un-tensioned state in accordance with the present disclosure. The paint roller 100 may be used by a user (not shown) to apply paint or coating over a surface, e.g., a wall. The paint roller 100 may include a roller assembly 102, a roller frame 104, a handle 106, and a roller cover (shown as roller cover 202 in FIG. 2).

The roller cover may be removably disposed on (e.g., inserted onto) the roller assembly 102. The roller cover may be configured to absorb paint and transfer the paint to the surface (e.g., a wall) quickly and evenly. In some aspects, the roller cover may be a cylindrical hollow core with an absorbent fabric at a roller cover exterior portion. The user may apply paint on the roller cover exterior portion and may “roll” the roller cover exterior portion on the surface to transfer the paint over the surface. The roller cover may be of any dimension. In some aspects, roller cover dimensions may be based on roller assembly dimensions.

The roller assembly 102 may include a first collar 108a and a second collar 108b. The first collar 108a may be disposed at a roller assembly proximal end and the second collar 108b may be disposed at a roller assembly distal end. The first collar 108a and the second collar 108b may be made of any material and may be of any size. For example, the first collar 108a and the second collar 108b may be made of plastic or metal, and may be a hollow cylinder with one cylinder end open (fully or partially) and second cylinder end closed. In other aspects, the first collar 108a may have both cylinder ends closed.

The roller assembly 102 may further include a guide rod 110 that may be disposed between the first collar 108a and the second collar 108b. The guide rod 110 may be an elongated tube or rod that may be cylindrical or cuboidal in shape. In some aspects, the guide rod 110 may be a hollow rod. In other aspects, the guide rod 110 may be a solid rod. The guide rod 110 may be made of plastic or metal (e.g., steel, aluminum, etc.) and have dimensions that depend on roller assembly dimensions or roller cover dimensions.

The guide rod 110 may include a guide rod proximal end and a guide rod distal end. The guide rod distal end may be coupled or attached to the second collar 108b. In an exemplary aspect, the guide rod distal end may be coupled to a second collar center portion. The guide rod distal end may be attached to the second collar 108b by using an adhesive or fastening means (not shown).

The guide rod proximal end may interact with a first collar center portion. For example, the guide rod proximal end may be inserted into a through-hole (not shown) disposed at the first collar center portion. In some aspects, the guide rod 110 may be configured to slide laterally via the through-hole of the first collar center portion to change a distance between the first collar 108a and the second collar 108b. Specifically, when the guide rod proximal end may “pulled” via the through-hole, the guide rod 110 may pull the second collar 108b towards the first collar 108a, thereby reducing the distance between the first collar 108a and the second collar 108b (reduced distance “D1” is shown in FIG. 1A). Similarly, when the guide rod proximal end may be “pushed” via the through-hole, the guide rod 110 may push the second collar 108b away from the first collar 108a, thereby increasing the distance between the first collar 108a and the second collar 108b (increased or extended distance “D2” is shown in FIG. 1B). The distance “D2” may be greater than the distance “D1”.

The roller assembly 102 may further include a plurality of deformable rods 112 that may be disposed between the first collar 108a and the second collar 108b. The plurality of deformable rods 112 may deform “outwards” away from the guide rod 110 when the distance between the first collar 108a and the second collar 108b may be decreased, as shown in FIG. 1A. The plurality of deformable rods 112 may move to be parallel to the guide rod 110 when the distance between the first collar 108a and the second collar 108b may be increased, as shown in FIG. 1B.

The plurality of deformable rods 112 may be configured to secure the roller cover on the roller assembly 102. For example, when the user inserts the roller cover onto the roller assembly 102, the user may cause the distance between the first collar 108a and the second collar 108b to be decreased, thereby deforming the plurality of deformable rods 112 in the outward direction. The “outwardly-deformed” rods may securely contact roller cover interior surface and lock or secure the roller cover over the roller assembly 102. An example view of a roller cover 202 secured over the roller assembly 102 is depicted in FIG. 2.

In some aspects, the plurality of deformable rods 112 may be attached to a first collar periphery portion and a second collar periphery portion. Stated another way, the plurality of deformable rods 112 may be configured to “surround” the guide rod 110, and may be disposed at a predetermined distance from the guide rod 110. A deformable rod longitudinal axis may be parallel to a guide rod longitudinal axis when the deformable rod may not be deformed. For example, as shown in FIG. 1B, when the distance between the first collar 108a and the second collar 108b may be a maximum permissible distance (e.g., the distance “D2”), the plurality of deformable rods 112 may be parallel to the guide rod 110. In some aspects, a length of each deformable rod may be equivalent to a guide rod length. In further aspects, the plurality of deformable rods 112 may be made of same material or different material as the guide rod 110. For example, the plurality of deformable rods 112 may be made of plastic.

The plurality of deformable rods 112 may be flexible rods that may be configured to deform when a pressure may be applied or when a tension may be created rod ends. Specifically, the plurality of deformable rods 112 may be configured to move between a tensioned state and an un-tensioned state based on the pressure applied between the rod ends. In the tensioned state, the plurality of deformable rods 112 may be configured to expand or move outwards (as depicted in FIG. 1A and described above), thereby securing or locking the roller cover on the roller assembly 102. In the un-tensioned state, the plurality of deformable rods 112 may be configured to “contract” or move inwards (as depicted in FIG. 1B and described above), thereby releasing or unlocking the roller cover from the roller assembly 102. The deformable rods 112 may be parallel to the guide rod 110 when the deformable rods 112 may be in the un-tensioned state.

As described above, the paint roller 100 may include the roller frame 104 and the handle 106. In some aspects, the roller frame 104 may be of any shape and may be made of any material. For example, the roller frame 104 may have a shape of a “question mark”, and the roller frame 104 may be made of metal or plastic. In some aspects, the roller frame 104 may be a hollow frame having a cavity that may be disposed throughout a frame length.

The roller frame 104 may include a first roller frame end and a second roller frame end. The first roller frame end may be attached to the first collar 108a (specifically a proximal closed-end surface of the first collar 108a), and the second roller frame end may be attached to the handle 106 (specifically a handle top end). The handle 106 may be of any shape and may be made of any material. For example, the handle 106 may be made of plastic. In some aspects, the handle 106 may be telescopic and may have adjustable height.

In some aspects, the paint roller 100 may further include a trigger mechanism that may facilitate the user to move the plurality of deformable rods 112 between the tensioned state and the un-tensioned state. Specifically, the trigger mechanism may be configured to change a distance between the first collar 108a and the second collar 108b to move the plurality of deformable rods 112 between the tensioned state and the un-tensioned state, based on user inputs or actuation.

In some aspects, the trigger mechanism may include a cable line 114 and a trigger lever 116. The cable line 114 may be disposed inside the roller frame 104 (that may be hollow, as described above) and the trigger lever 116 may be disposed at the handle 106 (e.g., the trigger lever 116 may be pivotally attached to a handle exterior surface). The cable line 114 may include a first cable end and a second cable end. The first cable end may be attached to the guide rod 110 proximal end, and the second cable end may be attached to the trigger lever 116. In some aspects, the trigger lever 116 may be configured to rotate and “pull” the cable line 114 to pull and slide the guide rod 110 proximal end via the through-hole located in the first collar 108a. For example, the user may rotate the trigger lever 116 to pull the cable line 114, thereby causing a change in distance (specifically decreasing the distance) between the first collar 108a and the second collar 108b. As described above, decreasing the distance between the first collar 108a and the second collar 108b may cause the plurality of deformable rods 112 to deform “outwards” (or switch from the un-tensioned state to the tensioned state).

In operation, the user may position the roller cover 202 on the roller assembly 102. Specifically, the user may slide the roller cover 202 over the roller assembly 102. The user may then rotate (e.g., in a first predefined direction, which may be clockwise direction) the trigger lever 116 to pull the cable line 114, thereby causing the plurality of deformable rods 112 to deform “outwards”. Specifically, when the user rotates the trigger lever 116, the trigger lever 116 may pull the second cable end (attached to the trigger lever 116), thereby pulling the first cable line and the guide rod 110 that may be connected to the first cable end. When the guide rod 110 may be pulled, the second collar 108b may move towards the first collar 108a, thereby moving the plurality of deformable rods 112 in the tensioned state. In the tensioned state, the plurality of deformable rods 112 may deform outwards (as depicted in FIG. 1A) to secure the roller cover 202 on the roller assembly 102, as shown in FIG. 2. In some aspects, the paint roller 100 may be in a “locked” state when the plurality of deformable rods 112 is deformed outwards. The user may use the paint roller 100 to paint a surface when the paint roller 100 may be in the locked state. For example, the user may apply paint one the roller cover outer surface, and then move or roll the “wet” roller cover 202 over the surface to transfer the paint from the roller cover 202 to the surface.

When the user may have completed the paint job or when the user desires to replace the roller cover 202, the user may use the trigger lever 116 to release the roller cover 202 from the roller assembly 102 without touching the roller cover 202. For example, the user may use (e.g., rotate in a second predefined direction, which may be counterclockwise direction) the trigger lever 116 to release the cable line 114 or move the paint roller 100 to an “unlocked” state. Releasing the cable line 114 may decrease or release the tension in the plurality of deformable rods 112 and may move the plurality of deformable rods 112 from the tensioned state to the un-tensioned state, as depicted in FIG. 1B. Specifically, releasing the cable line 114 may increase the distance between the first collar 108a and the second collar 108b. When the distance between the first collar 108a and the second collar 108b may be increased, the plurality of deformable rods 112 may move to be parallel to the guide rod 110, thereby facilitating the roller cover 202 to move or slide out of the roller assembly 102. In this manner, the user may cause the roller cover 202 to move out of the roller assembly 102, without requiring to touch the roller cover 202 during the removal process.

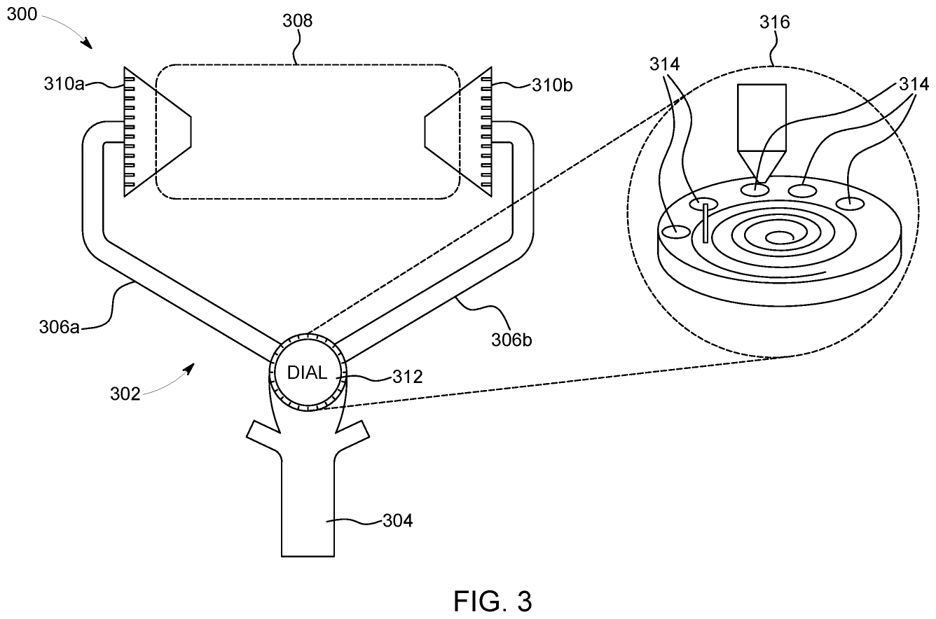

FIG. 3 depicts another paint roller 300 in accordance with the present disclosure. The paint roller 300 may include a roller frame 302 and a handle 304. The roller frame 302 may be of any shape and may be made of any material. In an exemplary aspect, the roller frame 302 may be “Y-shaped”, and may be made of plastic or metal.

The roller frame 302 may include a first arm 306a and a second arm 306b. Each arm may include a first end and a second end. First ends of both the first arm 306a and the second arm 306b may be configured to hold a roller cover 308 (that may be same as the roller cover 202) between the first arm 306a and the second arm 306b. Second ends of both the first arm 306a and the second arm 306b may be connected to the handle 304, via a trigger mechanism 312. The details of the trigger mechanism 312 is described later in the description below.

In some aspects, one arm (e.g., the first arm 306a) may be a stationary arm and the other arm (e.g., the second arm 306b) may be a “moveable” arm that may be configured to move closer to or away from the first arm 306a. The second arm 306b may be configured to move to facilitate easy removal and replacement of the roller cover 308 from the paint roller 300. In other aspects, both the first arm 306a and the second arm 306b may be moveable arms. In yet another aspect, the second arm 306b may be the stationary arm and the first arm 306a may be the moveable arm.

The paint roller 300 may further include a first support member 310a and a second support member 310b that may be configured to apply pressure on the roller cover 308 to secure the roller cover 308 between the first arm 306a and the second arm 306b. For example, the first support member 310a and the second support member 310b may be configured to apply pressure on roller cover 308 side edges to support the roller cover 308 and secure the roller cover 308 between the first and second arms 306a, 306b. The first support member 310a may be attached to the first arm 306a and the second support member 310b may be attached to the second arm 306b, for example, by using a fastening member (not shown). In some aspects, the roller cover 308 may be released when the second arm 306b may be moved away from the first arm 306a, thereby moving the second support member 310b. The movement of the second support member 310b may decrease/remove the pressure from the roller cover side edge, thereby facilitating easy removal/replacement of the roller cover 308 from the paint roller 300.

In some aspects, the first support member 310a and the second support member 310b may be axially rotatable that may enable the roller cover 308 to roll and paint the surface (e.g., wall surface) when the roller cover 308 may be secured between the first support member 310a and the second support member 310b.

The first support member 310a and the second support member 310b may be of any shape and may be made of any material. For example, both the first support member 310a and the second support member 310b may be conical in shape that may be configured to support the roller cover 308 of any dimension. Stated another way, the first support member 310a and the second support member 310b may have a tapered shape to hold the roller cover 308 of any dimension. In addition, the both the first support member 310a and the second support member 310b may be made of flexible material such as foam. In other aspects, the first support member 310a and the second support member 310b may be made of plastic or metal.

The paint roller 300 may include the trigger mechanism 312 configured to lock or unlock the roller cover 308 between the first and second arms 306a, 306b. Specifically, the trigger mechanism 312 may be configured to move the second arm 306b (e.g., the moveable arm) towards or away from the roller cover 308, thereby increasing or decreasing pressure on the roller cover 308 (e.g., by attaching/detaching the second arm 306b). In some aspects, the trigger mechanism 312 may include a dial index mechanism includes predetermined indexable positions 314, shown in view 316. The dial index mechanism may be configured to progressively add or release roller holding pressure on the roller cover 308 when actuated. In some aspects, actuating the dial index mechanism causes the second arm 306b to move and increase pressure or release pressure on the roller cover 308 (e.g., to secure or release the roller cover 308).

In operation, the user may actuate the dial index mechanism (e.g., rotate in anticlockwise direction) to move the second arm 306b towards the roller cover 308 to secure the roller cover 308. Moving the second arm 306b towards the roller cover 308 increases holding pressure on the roller cover 308, thus securing the roller cover 308. The user may select an indexable position for the dial index mechanism to apply appropriate holding pressure. When the user may have completed the paint job or when the user desires to replace the roller cover 202, the user may actuate the dial index mechanism (e.g., in clockwise direction) to move the second arm 306b away from the roller cover 308 to release the roller cover 308, without requiring to touch the roller cover 308 during the removal process. Moving the second arm 306b away from the roller cover 308 decreases holding pressure on the roller cover 308, thus facilitating easy removal of the roller cover 308.

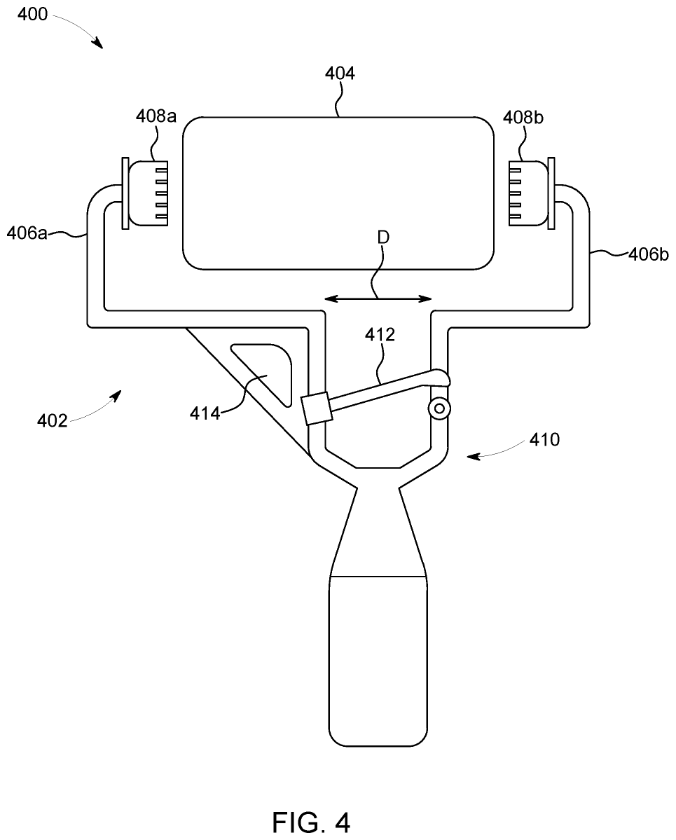

FIG. 4 depicts another paint roller 400 in accordance with the present disclosure. The paint roller 400 may include a roller frame 402 and a roller cover 404. The roller cover 404 may be same as the roller cover 202 and 308. The roller frame 402 may be made of any shape and may be made of any material. For example, the roller frame 402 may be made of plastic or metal.

The roller frame 402 may include a first arm 406a and a second arm 404b that may be connected to opposite ends of the roller cover 404. In an exemplary aspect, each of the first arm 404a and the second arm 404b may be shaped as a “question mark”. In some aspects, a first end of the first arm 306a may be connected to the roller cover 404 via a first support member 408a, and a first end of the second arm 306b may be connected to the roller cover 404 via a second support member 408b. The first support member 408a and the second support member 408b may be same as the first support member 310a and the second support member 310b as described in conjunction with FIG. 3, which may be configured to apply pressure on the roller cover 404 to secure the roller cover 404 on the roller frame 402. In some aspects (as shown in FIG. 4), the first support member 408a and the second support member 408b may be cylindrical in shape. In other aspects (not shown), the first support member 408a and the second support member 408b may be conical in shape. In some aspects, the first support member 408a and the second support member 408b may be axially rotatable that may enable the roller cover 404 to roll and paint the surface (e.g., wall surface) when the roller cover 404 may be secured between the first support member 408a and the second support member 408b.

A second end of both the first arm 306a and the second arm 306b may be connected to a connection unit 410 such that the second end of the first arm 306a and the second end of the second arm 306b may be disposed at a predetermined distance “D” from each other, as depicted in FIG. 4. The connection unit 410 may include a latching mechanism 412 (such as a lever 412) that may be configured to fasten the first arm 306a and the second arm 306b (via respective second ends). In some aspects, the lever 412 may be hinged at the second end of one arm (e.g., the first arm 406a, as depicted in FIG. 4). The lever 412 may be configured to move between a locking position and an unlocking position. In some aspects, the lever 412 may be in the locked position to secure the roller cover 404 in the paint roller 400, and the lever 412 may be in the unlocked position to facilitate easy removal/replacement of the roller cover 404.

The paint roller 400 may include a trigger mechanism 414 that may be configured to actuate the lever 412. The trigger mechanism 414 may be a finger-actuated mechanism that a user may operate using user fingers.

In operation, when the user triggers the trigger mechanism 414, the trigger mechanism 414 may actuate the lever 412. For example, when the user uses the trigger mechanism 414, the latching lever 412 may open which may “detach” the first arm 406a and the second arm 406b. Detaching the first arm 406a and the second arm 406b may increase the distance between the first support member 408a and the second support member 408b, thereby facilitating easy removal/replacement of the roller cover 404. For example, detaching the first arm 406a and the second arm 406b may decrease pressure applied to the roller frame 404 by the first support member 408a and the second support member 408b, which may facilitate easy removal of the roller cover 404 from the paint roller 400, without requiring the user to touch the roller cover 404 during the removal process.

In a similar manner, when the user desires to dispose a new roller cover in the paint roller 400, the user may position the new roller cover in between the first and second support members 408a, 408b, and may move the lever 412 in the locked position to secure the roller cover 404 in the paint roller 400. In this manner, the paint roller 400 may facilitate the user to conveniently dispose/remove a roller cover to/from the paint roller 400.

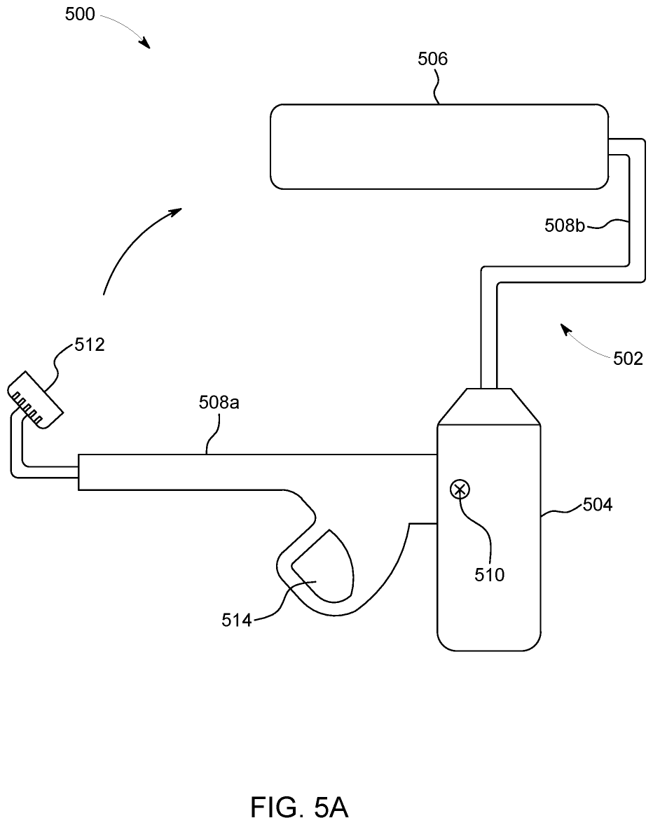

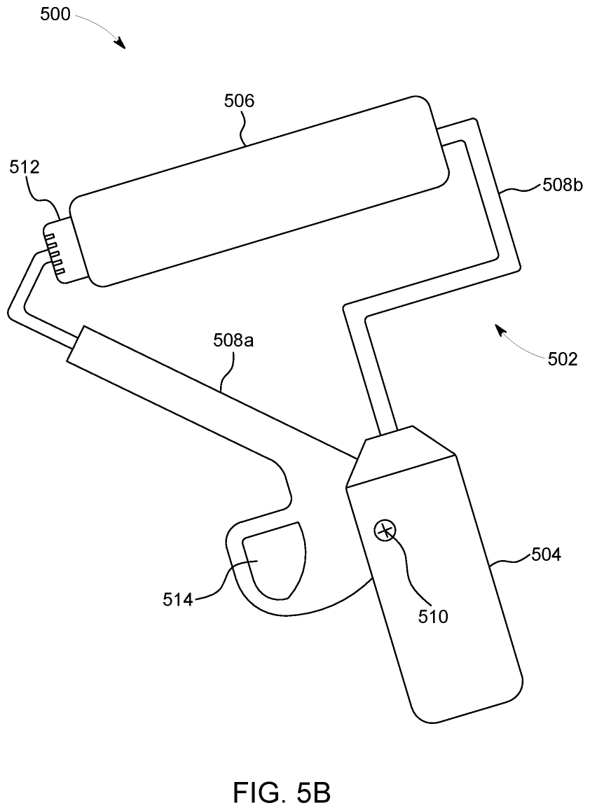

FIG. 5A depicts another paint roller 500 in an unlocked state and FIG. 5B depicts the paint roller 500 in a locked state, in accordance with the present disclosure.

The paint roller 500 may include a roller frame 502, a handle 504, and a roller cover 506. The roller frame 502 may be same as the roller frame 104, the handle 504 may be same as the handle 106, and the roller cover 506 may be same as the roller cover 202, 308, 404.

The roller frame 502 may include a first arm 508a and a second arm 508b. In some aspects, the first arm 508a may be a moveable arm and the second arm 508b may a stationary arm, as depicted in FIG. 5A. In some aspects, one end of the second arm 508b may be attached to the handle 504 (e.g., using any fastening mechanism or adhesive) and the other end of the second arm 508b may be attached to the roller cover 506. Further, one end of the first arm 508a may be pivotally attached to the handle 504 by using a pivotal connection 510 disposed on the handle 504. The other end of the first arm 508a may include a support member 512 that may be configured to contact a roller cover side edge and provide pressure to the roller cover 506 to secure the roller cover 506 on the roller frame 502, as shown in FIG. 5B. The support member 512 may be same as the support members described above, e.g., support members 310a, 310b, 408a, 408b.

The first arm 508a may further include a trigger mechanism 514 that may be configured to move the first arm 508a between a “locked” state and an “unlocked” state. FIG. 5A depicts the first arm 508a in the un-locked state in which the first arm 508a may be detached from the roller cover 506 (e.g., the support member 512 may not contact the roller cover 506). The user may easily remove/replace the roller cover 506 without requiring to touch the roller cover 202 during the removal process when the first arm 508a may be in the unlocked state. FIG. 5B depicts the first arm 508a in the locked state in which the first arm 508a (specifically the support member 512) may be attached to or contact the roller cover 506 to secure the roller cover 506 in the paint roller 500. In some aspects, the user may actuate the trigger mechanism 514 to move the first arm 508a between the locked state and the unlocked state.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc., should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. An apparatus comprising:
a roller assembly comprising:
a first collar disposed at a roller assembly proximal end;
a second collar disposed at a roller assembly distal end;
a guide rod having a guide rod proximal end and a guide rod distal end, wherein the guide rod distal end is attached to the second collar, and wherein the guide rod is disposed between the first collar and the second collar; and
a plurality of deformable rods disposed between the first collar and the second collar, wherein the plurality of deformable rods is configured to move between a tensioned state and an un-tensioned state, and wherein the plurality of deformable rods is configured to secure a roller cover on the roller assembly in the tensioned state; and
a trigger mechanism configured to move the plurality of deformable rods between the tensioned state and the un-tensioned state, wherein the trigger mechanism comprises a cable line connected to the guide rod proximal end.

2. The apparatus of claim 1, wherein the guide rod distal end is attached to a second collar center portion.

3. The apparatus of claim 1, wherein the guide rod proximal end is configured to be inserted into a through-hole located at a first collar center portion.

4. The apparatus of claim 3, wherein the trigger mechanism is configured to move the guide rod proximal end via the through-hole to change a distance between the first collar and the second collar, and wherein the trigger mechanism changes the distance to move the plurality of deformable rods between the tensioned state and the un-tensioned state.

5. The apparatus of claim 4 further comprising a roller frame having a first roller frame end and a second roller frame end, wherein the first roller frame end is attached to the first collar.

6. The apparatus of claim 5 further comprising a handle, wherein the second roller frame end is attached to the handle.

7. The apparatus of claim 6, wherein the cable line is disposed inside the roller frame.

8. The apparatus of claim 7, wherein the trigger mechanism comprises a trigger lever that is disposed at the handle.

9. The apparatus of claim 8, wherein the trigger lever is configured to move the cable line to slide the guide rod proximal end laterally via the through-hole.

10. The apparatus of claim 1, wherein the plurality of deformable rods is coupled to a first collar periphery portion and a second collar periphery portion.

11. An apparatus comprising:
a roller assembly comprising:
a first collar disposed at a roller assembly proximal end;
a second collar disposed at a roller assembly distal end;
a guide rod having a guide rod proximal end and a guide rod distal end, wherein the guide rod distal end is attached to the second collar, and wherein the guide rod is disposed between the first collar and the second collar; and
a plurality of deformable rods disposed between the first collar and the second collar, wherein the plurality of deformable rods is configured to move between a tensioned state and an un-tensioned state, and wherein the plurality of deformable rods is configured to secure a roller cover on the roller assembly in the tensioned state; and
a trigger mechanism configured to move the plurality of deformable rods between the tensioned state and the un-tensioned state, wherein the trigger mechanism comprises a cable line connected to the guide rod proximal end, and wherein the cable line is configured to move the guide rod proximal end to change a distance between the first collar and the second collar.

12. The apparatus of claim 11, wherein the guide rod distal end is attached to a second collar center portion.

13. The apparatus of claim 11, wherein the guide rod proximal end is configured to be inserted into a through-hole located at a first collar center portion.

14. The apparatus of claim 13, wherein the cable line is configured to move the guide rod proximal end via the through-hole to change the distance between the first collar and the second collar, and wherein the cable line changes the distance to move the plurality of deformable rods between the tensioned state and the un-tensioned state.

15. The apparatus of claim 14 further comprising a roller frame having a first roller frame end and a second roller frame end, wherein the first roller frame end is attached to the first collar.

16. The apparatus of claim 15 further comprising a handle, wherein the second roller frame end is attached to the handle.

17. The apparatus of claim 16, wherein the cable line is disposed inside the roller frame.

18. The apparatus of claim 17, wherein the trigger mechanism comprises a trigger lever that is disposed at the handle.

19. The apparatus of claim 18, wherein the trigger lever is configured to move the cable line to slide the guide rod proximal end laterally via the through-hole.

20. An apparatus comprising:
a roller assembly comprising:
a first collar disposed at a roller assembly proximal end;
a second collar disposed at a roller assembly distal end;
a guide rod disposed between the first collar and the second collar; and
a plurality of deformable rods disposed between the first collar and the second collar, wherein the plurality of deformable rods is configured to move between a tensioned state and an un-tensioned state, and wherein the plurality of deformable rods is configured to secure a roller cover on the roller assembly in the tensioned state; and
a trigger mechanism configured to change a distance between the first collar and the second collar to move the plurality of deformable rods between the tensioned state and the un-tensioned state.
